(19) United States
(12) Patent Application Publication
(10) Pub. No.: US 2011/0034424 A1
(43) Pub. Date: Feb. 10, 2011

(54) METHOD FOR THE LONG TERM NSAID USE

(75) Inventor: Ryuji Ueno, Montgomery, MD (US)

Correspondence Address: SUGHRUE MION, PLLC, 2100 PENNSYLVANIA AVENUE, N.W., SUITE 800, WASHINGTON, DC, 20037, US

(73) Assignee: SUCAMPO AG, Zug (CH)

(21) Appl. No.: 12/826,108

(22) Filed: Jun. 29, 2010

Related U.S. Application Data
(60) Provisional application No. 61/221,698, filed on Jun. 30, 2009.

(51) Int. Cl. A61K 31/60; A61K 31/405; A61K 31/353; A61K 31/407; A61K 31/5415; A61K 31/4152; A61K 31/416; A61K 31/515; A61K 31/47; A61K 31/44; A61K 31/381; A61P 29/00

(52) U.S. Cl. 514/161; 514/420; 514/456; 514/413; 514/224.2; 514/411; 514/404; 514/405; 514/270; 514/311; 514/352; 514/448

(57) ABSTRACT

Provided is a method for the long term treatment of a condition or disease which is one of the indications for NSAID use in a human patient, which comprises administering to the patient a pharmaceutically effective amount of a NSAID and at least 36 mcg per day of cobiprostone or its pharmaceutically acceptable salt, ester, ether or amide. By administering cobiprostone or its pharmaceutically acceptable salt, ester, ether or amide in combination with a NSAID, the patient can receive the NSAID for longer time period.

### CROSS REFERENCES TO THE RELATED APPLICATIONS

This application claims priority from a provisional application filed Jun. 30, 2009 and assigned Ser. No. 61/221,698. The whole contents of the provisional application are herein incorporated by reference.

### TECHNICAL FIELD

The present invention relates to a method or a medicament that enables long-term NSAID therapy.

### BACKGROUND ART

Non-steroidal anti-inflammatory drugs (NSAIDs) are among the most commonly used drugs worldwide. Although the analgesic, anti-pyretic and anti-inflammatory properties of NSAIDs are very effective for the treatment of pain and inflammation, long term use of a NSAID can cause gastrointestinal injury ranging from upset stomach to ulcer formation and gastrointestinal bleeding. Due to those adverse side effects, NSAIDs are recommended for short term use.

Cobiprostone is a functional fatty acid and a member of a class of compounds called prostones. It is a locally acting chloride channel activator that works on ion channels located in the liver and the gastrointestinal tract. In animal studies, cobiprostone protected against formation of ulcers induced by indomethacin, an NSAID, and ulcers induced by stress and demonstrated an acceptable safety.

U.S. Pat. Nos. 5,225,439, 5,166,174, 5,284,858, 5,428,062, 5,380,709, 5,886,034 and 6,265,440 describe that certain prostaglandin E compounds are effective for the treatment of ulcers such as duodenal ulcer and gastric ulcer.

U.S. Pat. No. 7,064,148 describes prostaglandin compound opens and activates chloride channels, especially ClC channels, more especially ClC-2 channel.

U.S. Patent publication No. 2003/0166632 describes ClC-2 channel opener is effective for the treatment of a disease or a condition responsive to opening of ClC-2 channel.

U.S. Patent publication No. 2009/0012165 describes a pharmaceutical combination comprising a NSAID and a specific prostaglandin compound.

However, it is not known how cobiprostone acts on the long term NSAID use.

### DISCLOSURE OF THE INVENTION

An object of the present invention to provide a method or medicament for treating one of indications for NSAID use that enable long-term administration of a NSAID without discontinuation of NSAID use, for example, with reduced risk of side effects.

The present invention relates to a method for the long term treatment of a condition or disease which is one of the indications for NSAID use in a human patient, which comprises administering to the patient a pharmaceutically effective amount of a NSAID and at least 36 mcg per day of cobiprostone or its pharmaceutically acceptable salt, ester, ether or amide.

The present invention also provides a medicament comprising cobiprostone or its pharmaceutically acceptable salt, ester, ether or amide, for the long term treatment of a disease or condition which is one of indications for NSAID use in a human patient, wherein cobiprostone is administered at least 36 mcg per day to the patient who receives a NSAID.

The present invention further provides a combination for the long term treatment of a condition or disease which is one of the indications for NSAID use in a human patient, comprising a pharmaceutically effective amount of a NSAID and cobiprostone or its pharmaceutically acceptable salt, ester, ether or amide, wherein cobiprostone is administered at least 36 mcg per day.

Further more, the present invention further provides use of an effective amount of a NSAID and cobiprostone or its pharmaceutically acceptable salt, ester, ether or amide, for manufacturing a medicament for the long term treatment of a condition or disease which is one of the indications for NSAID use in a human patient, wherein cobiprostone is administered at least 36 mcg per day.

By administering cobiprostone or its pharmaceutically acceptable salt, ester, ether or amide in combination with a NSAID, the gastrointestinal injury caused by the administration of the NSAID is well suppressed and the patient can receive the NSAID for longer time period, for example, at least 4 weeks, at least 8 weeks, or for at least 12 weeks.

### DETAILED DESCRIPTION OF THE INVENTION

The NSAID used in the present invention may be selected from but not limited to the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prenazone, apazone, benzydamine, bucolome, cinchophen, clonixin, ditrazol, epirizole, fenoprofen, floctafenin, flufenamic acid, glaphenine, indoprofen, ketoprofen, loxoprofen, meclofenamic acid, mefenamic acid, niflumic acid, phenacetin, salidifamides, sulindac, suprofen, tolmetin, pharmaceutically acceptable salts thereof, and mixtures thereof. The preferable example of NSAID is naproxen.

Cobiprostone, a functional fatty acid and a member of a class of compounds called prostones, has a chemical name of 7{(2R,4aR,5R,7aR)-2-[(3S)-1,1-difluoro-3-methylpentyl]-2-hydroxy-6-oxooctahydrocyclopenta[b]pyran-5-yl}heptanoic acid.

Suitable “pharmaceutically acceptable salt” of cobiprostone include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Example of the ether include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Example of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide means a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Cobiprostone of this invention exists as a bicyclic form in a solid state, but partially forms a tautomer of it when dissolved in a solvent. In the absence of water, cobiprostone exists predominantly in the form of the bicyclic compound. In aqueous media, it is believed that hydrogen bonding occurs between, for example, the ketone position at the C-15 position, thereby hindering bicyclic ring formation.

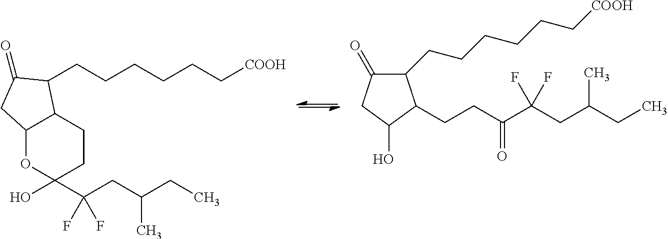

According to the present invention, said “tautomer” of cobiprostone, may also be used for the treatment.

The cobiprostone or its salt, ether ester or amide used in the present invention may be prepared by the method disclosed in U.S. Pat. No. 5,739,161 (this cited reference is herein incorporated by reference).

According to the present invention, the NSAID and cobiprostone are both administered to a patient simultaneously in the form of a single entity or dosage, or are both administered to a patient as separate entities either simultaneously or sequentially with no specific time limits, wherein such administration provides therapeutically effective levels of the two components in the body, preferably at the same time.

According to the present invention, each of cobiprostone or its salt, ether, ester or amide and the NSAID used in combination with the cobiprostone or its salt, ether, ester or amide may be applied systemically or topically. Usually, the compound may be administered by oral administration, intravenous injection (including infusion), subcutaneous injection, intranasal administration, inhalational administration, intra rectal administration, intra vaginal administration, transdermal administration and the like. The dose may vary depending on the strain of the animal, age, body weight, symptom to be treated, desired therapeutic effect, administration route, term of treatment and the like. A satisfactory effect can be obtained by systemic administration 1-4 times per day or continuous administration at the amount of at least 36 mcg, more preferably at least 54 mcg of cobiprostone, and 0.01-100000 mg, preferably 0.1-10000 mg, preferably 1-1000 mg of a NSAID at a daily dose.

According to the invention, the cobiprostone or its salt, ether ester or amide and the NSAID used in combination with cobiprostone or its salt, ether ester or amide may be formulated in any form with a pharmaceutically acceptable excipient. The pharmaceutically suitable excipient may be, therefore, selected depending on the desired form of the composition. According to the invention, “pharmaceutically suitable excipient” means an inert substance, which is suitable for the form, combined with the active ingredient of the invention.

For example, solid composition for oral administration of the present invention may include tablets, preparations, granules and the like. In such a solid composition, one or more active ingredients may be mixed with at least one inactive diluent, for example, lactose, mannitol, glucose, hydroxypropyl cellulose, microcrystalline cellulose, starch, polyvinyl pyrrolidone, magnesium aluminate metasilicate and the like. According to the usual work-up, the composition may contain additives other than inactive diluent, for example, lubricant such as magnesium stearate; disintegrant such as fibrous calcium gluconate; stabilizer such as cyclodextrin, for example, α,β- or γ-cyclodextrin; etherified cyclodextrin such as dimethyl-α-, dimethyl-β-, trimethyl-β-, or hydroxypropyl-β-cyclodextrin; branched cyclodextrin such as glucosyl-, maltosyl-cyclodextrin; formylated cyclodextrin, cyclodextrin containing sulfur; phospholipid and the like. When the above cyclodextrins are used, an inclusion compound with cyclodextrins may be sometimes formed to enhance stability. Alternatively, phospholipid may be sometimes used to form a liposome, resulting in enhanced stability.

Tablets or pills may be coated with film soluble in the stomach or intestine such as sugar, gelatin, hydroxypropyl cellulose, or hydroxypropylmethyl cellulose phthalate as needed. Further, the compounds may be formed as capsules with absorbable substances such as gelatins. Preferably, the cobiprostone or its salt, ether, ester or amide is formulated in a soft gelatin capsule with a medium chain fatty acid triglyceride. Examples of the medium chain fatty acid triglyceride used in the present invention include a triglyceride of a saturated or unsaturated fatty acid having 6-14 carbon atoms which may have a branched chain. A preferred fatty acid is a straight chain saturated fatty acid, for example caproic acide (C6), caprylic acid (C8), capric acid (C10), lauric acid (C12) and myristic acid (C14). In addition, two or more medium chain fatty acid triglycerides may be used in combination. Further suitable excipients are disclosed in U.S. Pat. No. 6,583,174.

A liquid composition for oral administration may be pharmaceutically acceptable emulsion, solution, suspension, syrup, or elixir, as well as generally used inactive diluent. Such composition may contain, in addition to the inactive diluent, adjuvants such as lubricants and suspensions, sweetening agents, flavoring agents, preservatives, solubilizers, anti-oxidants and the like. The details of the additives may be selected from those described in any general textbooks in the pharmaceutical field. Such liquid compositions may be directly enclosed in soft capsules. Solutions for parenteral administration, for example, suppository, enema and the like according to the present invention include sterile, aqueous or non-aqueous solution, suspension, emulsion, detergent and the like. The aqueous solution and suspension includes, for example, distilled water, physiological saline and Ringer's solution.

The composition of the present invention may be in the form of spraying composition, which contains one of more active ingredients and may be prepared according to a known method.

Example of the intranasal preparations may be aqueous or oily solutions, suspensions or emulsions comprising one or more active ingredient. For the administration of an active ingredient by inhalation, the composition of the present invention may be in the form of suspension, solution or emulsion which can provide aerosol or in the form of powder suitable for dry powder inhalation. The composition for inhalational administration may further comprises a conventionally used propellant.

The non-aqueous solution and suspension include, for example, propylene glycol, polyethylene glycol, fatty acid triglyceride, and vegetable oil such as olive oil, alcohols such as ethanol, polysorbate and the like. Such composition may contain adjuvants such as preservatives, wetting agent, emulsifier, dispersant, anti-oxidants and the like.

Examples of the injectable compositions of the present invention for parenteral administration include sterile aqueous or non-aqueous solutions, suspensions and emulsions. Diluents for the aqueous solution or suspension may include, for example, distilled water for injection, physiological saline and Ringer's solution.

Non-aqueous diluents for solution and suspension may include, for example, propylene glycol, polyethylene glycol, vegetable oils such as olive oil, alcohols such as ethanol and polysorbate. The composition may further comprise additives such as preservatives, wetting agents, emulsifying agents, dispersing agents and the like. They may be sterilized by filtration through, e.g. a bacteria-retaining filter, compounding with a sterilizer, or by means of gas or radioisotope irradiation sterilization. The injectable composition may also be provided as a sterilized powder composition to be dissolved in a sterilized solvent for injection before use.

External agent includes all the external preparations, which includes ointment, cream, liquids, lotion, paste, patch and spray.

Another form of the medicament of the present invention is suppository or pessary, which may be prepared by mixing active ingredients into a conventional base such as cacao butter that softens at body temperature, and nonionic surfactants having suitable softening temperatures may be used to improve absorbability.

According to the method of the invention, the compound of the present invention can be administered systemically or locally by means of oral or parental administration, including a suppository, enema and the like as well as it may be an external agent. Single or multiple compositions may be administered to achieve the desired dose.

The term “treatment” used herein includes any means of control such as prevention, care, relief of the condition, attenuation of the condition and arrest of progression.

Cobiprostone or its pharmaceutically acceptable salt, ester, ether or amide can be used in combination with NSAID for the long term treatment of a condition or disease which is one of the indications for NSAID use. Examples of the indications for NSAID use may include arthritis, rheumatoid arthritis, osteoarthritis, spondylitis, ankylosing spondylitis, juvenile arthritis, juvenile rheumatoid arthritis, tendonitis, bursitis, gout, pain, and dysmenorrhea. The indications for NSAID use may further comprises Alzheimer disease or cancers.

The combination of cobiprostone or its pharmaceutically acceptable salt, ester, ether or amide and a NSAID is especially useful for the long term treatment of pain from various etiologies.

The term “pain from various etiology” includes, but is not limited to, inflammatory pain, hyperalgesia and, in particular, chronic pain, and means in particular pain consequential to trauma, e.g. associated with burns, sprains, fracture or the like, subsequent to surgical intervention, e.g. as post-operative analgesics, chemotherapy-induced pain, as well as inflammatory pain of diverse genesis, e.g. bone and joint pain (osteoarthritis), myofascial pain (muscular injury, fibromyalgia), lower back pain, chronic inflammatory pain, chronic neuropathic pain, e.g. diabetic neuropathy, phantom limb pain and perioperative pain (general surgery, gynecologic surgery) as well as pain associated with, e.g., angina, menstruation or cancer.

The term “cancer” includes esophageal cancer, gastric cancer, duodenal cancer, small intestinal cancer, appendiceal cancer, large bowel cancer, colon cancer, rectum cancer, colorectal cancer, anal cancer, pancreatic cancer, liver, cancer, gallbladder cancer, spleen cancer, renal cancer, bladder cancer, prostatic cancer, testicular cancer, uterine cancer, ovarian cancer, mammary cancer, pulmonary cancer and thyroid cancer.

The further details of the present invention will follow with reference to test examples, which, however, are not intended to limit the present invention.

### EXAMPLE

A total of 124 patients with osteoarthritis and/or rheumatoid arthritis were enrolled in 12-week, double-blinded, randomized, dose-ranging and placebo-controlled phase 2 trial. All patients in the trial received 500 mg of naproxen twice a day. There were four treatment cohorts: one cohort received placebo while the other three cohorts received 18 mcg of cobiprostone either once, twice or three times a day (daily totals of 18, 36 or 54 mcg, respectively).

Retention rate and withdrawal rate were evaluated by the following definitions.

Retention rate: The number of intent-to-treat subjects that remained in the trial at a specified week.

Withdrawal Rate: The number of randomized subjects that discontinued the trial. The reasons for discontinuation were:

a. Adverse event

b. Discontinued due to ulcer

c. Lost to follow-up

d. Non-compliance

e. Patient choice

f. Sponsor request

The withdrawal rate can be expressed/calculated for a single reason such as adverse events, or for any combination of the discontinuation reasons.

The retention rates of patients taking naproxen with cobiprostone at Week 8 were 50% for placebo vs. 67%, 66% and 90% for cobiprostone 18 mcg, 36 mcg and 54 mcg, respectively. The retention rates of patients taking naproxen with cobiprostone at Week 12 were statistically significant when compared to patients taking naproxen with placebo and increased in a dose-dependent manner. The rates were 40% for placebo vs. 47%, 52% and 77% for cobiprostone 18 mcg, 36 mcg and 54 mcg, respectively. The median number of days in the treatment period was 55.0 days for patients taking placebo compared to 60.0, 82.0, and 83.0 days for cobiprostone 18 mcg, 36 mcg and 54 mcg, respectively.

Overall, the data showed cobiprostone was well tolerated in patients receiving NSAID therapy. The related overall adverse event rates were 66.7% for placebo, compared to 60.0%, 71.0% and 67.7% for cobiprostone 18 mcg, 36 mcg and 54 mcg, respectively. The most common related adverse events were: diarrhea, at 13.3% for placebo compared to 13.3%, 32.3% and 35.5% for cobiprostone 18 mcg, 36 mcg and 54 mcg, respectively; nausea, at 10.0% for placebo vs. 10.0%, 16.1% and 16.1% for cobiprostone 18 mcg, 36 mcg and 54 mcg, respectively; and gastritis, at 13.3% for placebo vs. 13.3%, 6.5% and 9.7% for cobiprostone 18 mcg, 36 mcg and 54 mcg, respectively. The drug-related gastrointestinal adverse event rates were 66.7% for placebo vs. 60.0%, 67.7%, and 67.7% for cobiprostone 18 mcg, 36 mcg and 54 mcg, respectively, which suggest that gastrointestinal adverse events other than diarrhea and nausea may be related to the naproxen therapy. Withdrawal rates from the trial due to an adverse event were: 21.9% for placebo vs. 13.3%, 16.1% and, 16.1% for cobiprostone 18 mcg, 36 mcg and 54 mcg, respectively.

The above data indicates that at least 36 mcg, especially at least 54 mcg of cobiprostone per day is useful for the long term NSAID use. That is, by receiving said dose of cobiprostone in combination with a NSAID, the patient can be treated by the NSAID for longer period of time without discontinuation of NSAID use, for example, with reduced side effects.

What is claimed is:

1. A method for the long term treatment of a condition or disease which is one of the indications for NSAID use in a human patient, which comprises administering to the patient a pharmaceutically effective amount of a NSAID and at least 36 mcg per day of cobiprostone or its pharmaceutically acceptable salt, ester, ether or amide.

2. The method as described in claim 1, wherein the long term treatment is for at least 4 weeks.

3. The method as described in claim 1, wherein the long term treatment is for at least 8 weeks.

4. The method as described in claim 1, wherein the long term treatment is for at least 12 weeks.

5. The method as described in claim 1, wherein the NSAID is selected from the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prenazone, apazone, benzydamine, bucolome, cinchophen, clonixin, ditrazol, epirizole, fenoprofen, floctafenin, flufenamic acid, glaphenine, indoprofen, ketoprofen, loxoprofen, meclofenamic acid, mefenamic acid, niflumic acid, phenacetin, salidifamides, sulindac, suprofen, tolmetin, a pharmaceutically acceptable salt thereof, and a mixture thereof.

6. The method as described in claim 5, the NSAID is naproxen.

7. The method as described in claim 6, naproxen is administered at least 500 mg per day.

8. The method as described in claim 6, naproxen is administered at least 500 mg twice per day.

9. The method as described in claim 1, wherein cobiprostone is administered at least 54 mcg per day.

10. The method as described in claim 1, wherein the respective compounds are administered simultaneously, separately or sequentially.
